(19) United States
(12) Patent Application Publication
(10) Pub. No.: US 2024/0312618 A1
(43) Pub. Date: Sep. 19, 2024

(54) Method of automated intraoral x-ray acquisition

(71) Applicants: Douglas A. Golay, Coon Rapids, IA (US); Wyatt C. Davis, Bozeman, MT (US)

(72) Inventors: Douglas A. Golay, Coon Rapids, IA (US); Wyatt C. Davis, Bozeman, MT (US)

(21) Appl. No.: 18/122,611

(22) Filed: Mar. 16, 2023

(51) Int. Cl. G16H 40/63; A61B 6/14

(52) U.S. Cl. CPC G16H 40/63; A61B 6/512

(57) ABSTRACT

A computing device implemented method includes the step of using a third-party disparate dental imaging system with capabilities to directly control a dental intraoral x-ray sensor imaging device via enacting communication with that specific brand of dental intraoral x-ray sensor imaging device for the purpose of acquiring new dental intraoral x-ray images of a patient's dental anatomy. The computing device implemented method also includes the step of using a decoupled software application that is not part of the third-party dental imaging software and which decoupled software contains an algorithm that detects when a specific brand or version of third-party dental imaging software is enacted upon the same computing device as the decoupled software application is executing. The computing device implemented method automates acquisition of images from non-supported dental imaging devices into closed architecture dental imaging software.

### BACKGROUND OF THE INVENTION

### Field of the Invention

This present invention relates generally to dental imaging software and more particularly to automating acquisition of images from non-supported dental imaging devices into closed architecture dental imaging software.

### Description of the Prior Art

In the field of Dentistry digital intraoral x-ray imaging has become popular over the last twenty-five plus years and has nearly replaced all use of analog film in dental offices. Digital intraoral x-ray sensors require an imaging software to post-process, store, view, and communicate images produced by the digital x-ray sensor device. In the early user adoption-years of digital intraoral x-ray imaging, manufacturers of sensors also produced the imaging software and that imaging software was the only software that could operate that specific brand/model of x-ray sensor. This is referred to as a closed architecture system. As dental digital intraoral x-ray became more popular some imaging software's expanded to include limited inter-operability via using standards such as Twain drivers for the sensor and imaging software which facilities easy integration and no proprietary programming required to the imaging software to communicate with that specific brand/model of digital intraoral x-ray sensor. Twain has limitations in dental workflows for series acquisition and other, image quality/bit depth limitation, pre-processing limitations, and less robust error checking versus a directly supported x-ray imaging sensor device. As progress continued in dental digital x-ray system development some vendors now produce “open architecture” imaging software that can operate multiple models (brands) of sensors produced by different manufacturers via programming directly to that specific manufacturer sensor driver/software development kit (i.e., SDK). Open architecture allows maximum inter-operability within the same imaging software allowing support of multiple brands of sensors simultaneously and inter-changeably, but some digital x-ray sensor vendors do not provide drivers/SDKs to allow inter-operability of their sensor within a third-party open architecture imaging software. Users of original closed architecture imaging software's that do not allow third-party open architecture software to support their sensors leaves the dentist with the options of continuing to use only the sensor vendors imaging software and only with their brand of sensor. Not allowing expansion to use specific other brands or models of sensors within that same imaging software does not exist and which option is less than desirable to the dentist/care provider and limits flexibility and patient care options.

U.S. Patent Application Publication No. 2013/0226993 teaches a media acquisition engine which includes an interface engine that receives a selection from a plug-in coupled to a media client engine where a client associated with the media client engine identified as subscribing to a cloud application imaging service. The media acquisition engine also includes a media control engine that directs, in accordance with the selection, a physical device to image a physical object and produce a media item based on the image of the physical object, the physical device being coupled to a cloud client. The media acquisition engine also includes a media reception engine that receives the media item from the physical device and a translation engine that encodes the media item into a data structure compatible with the cloud application imaging service. The interface engine is configured to transfer the media item to the plug-in. Digital imaging has notable advantages over traditional imaging, which processes an image of a physical object onto a physical medium. Digital imaging help users such as health professionals avoid the costs of expensive processing equipment, physical paper, physical radiographs, and physical film. Techniques such as digital radiography expose patients to lower doses of radiation than traditional radiography and are often safer than their traditional counterparts are. Digital images are easy to store on storage such as a computer's hard drive or a flash memory card, are easily transferable and are more portable than traditional physical images. Many digital imaging devices use sophisticated image manipulation techniques and filters that accurately image physical objects. A health professional's information infrastructures and the business processes can therefore potentially benefit from digital imaging techniques. Though digital imaging has many advantages over physical imaging, digital imaging technologies are far from ubiquitous in health offices as existing digital imaging technologies present their own costs. To use existing digital imaging technologies, a user such as a health professional has to purchase separate computer terminals and software licenses for each treatment room. As existing technologies install a full digital imaging package on each computer terminal, these technologies are often expensive and present users with more options than they are willing to pay for. Additionally, existing digital imaging technologies require users to purchase a complete network infrastructure to support separate medical imaging terminals. Users often face the prospects of ensuring software installed at separate terminals maintains patient confidentiality, accurately stores and backs up data, accurately upgrades, and correctly performs maintenance tasks. Existing digital imaging technologies are not readily compatible with the Objectives of end-users, such as health professionals.

Referring to FIG. 1 a networking system 100 includes a desktop computer 102, a laptop computer 104, a server 106, a network 108, a server 110 a server 112, a tablet device 114 and a private network group 120 in order to provide at least one or more application imaging services. The private network group 120 includes a laptop computer 122, a desktop computer 124, a scanner 126, a tablet device 128, an access gateway 62, a first physical device 64, a second physical device 66 and a third physical device 68. The desktop computer 102, the laptop computer 104, the server 106, the server 110 the server 112, and the tablet device 114 are directly connected to the network 108. The desktop computer 102 may include a computer having a separate keyboard, a mouse, a display/monitor and a microprocessor. The desktop computer can integrate one or more of the keyboard, the monitor, and the processing unit into a common physical module. The laptop computer 104 can include a portable computer. The laptop 104 can integrate a keyboard, a mouse, a display/monitor and a microprocessor into one physical unit. The laptop 104 also has a battery so that the laptop 104 allows portable data processing and portable access to the network 108. The tablet 114 can include a portable device with a touch screen, a display/monitor, and a processing unit all integrated into one physical unit. Any or all of the computer 102, the laptop 104 and the tablet device 118 may include a computer system. A computer system will usually include a microprocessor, a memory, a non-volatile storage and an interface. Peripheral devices can also form a part of the computer system. A typical computer system will include at least a processor, memory, and a bus coupling the memory to the processor. The processor can include a general-purpose central processing unit (CPU), such as a microprocessor or a special-purpose processor, such as a microcontroller. The memory can include random access memory (RAM), such as dynamic RAM (DRAM) and static RAM (SRAM). The memory can be local, remote, or distributed. The term “computer-readable storage medium” includes physical media, such as memory. The bus of the computer system can couple the processor to non-volatile storage. The non-volatile storage is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. A direct memory access process often writes some of this data into memory during execution of software on the computer system. The non-volatile storage can be local, remote, or distributed. The non-volatile storage is optional because systems need only have all applicable data available in memory. Software is typically stored in the non-volatile storage. Indeed, for large programs, it may not even be possible to store the entire program in memory. Nevertheless, for software to run, if necessary, it is moved to a computer-readable location appropriate for processing. Even when software is moved to the memory for execution, the processor will make use of hardware registers to store values associated with the software, and local cache that, ideally, serves to speed up execution. A software program is assumed to be stored at any known or convenient location from non-volatile storage to hardware registers when the software program is referred to as “implemented in a computer-readable storage medium.” A microprocessor is “configured to execute a program” when at least one value associated with the program is stored in a register readable by the microprocessor. The bus can also couple the microprocessor to one or more interfaces. The interface can include one or more of a modem or network interface. A modem or network interface can be part of the computer system. The interface can include an analog modem, ISDN modem, cable modem, token ring interface, satellite transmission interface or other interfaces for coupling a computer system to other computer systems. The interface can include one or more input and/or output (I/O) devices. The I/O devices can include a keyboard, a mouse or other pointing device, disk drives, printers, a scanner, and other I/O devices, including a display/monitor. The display/monitor device can include a cathode ray tube (CRT), liquid crystal display (LCD) or some other applicable known or convenient display device. Operating system software includes a file management system, such as a disk operating system, can control the computer system. One operating system software with associated file management system software is the family of operating systems known as Windows from Microsoft Corporation of Redmond, Wash., and their associated file management systems. Another operating system software with its associated file management system software is the Linux operating system and its associated file management system. The file management system is typically stored in the non-volatile storage and causes the processor to execute the various acts required by the operating system to input and output data and to store data in the memory, including storing files on the non-volatile storage. Some portions of the detailed description refer to algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. All of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices. The algorithms and displays presented herein do not inherently relate to any particular computer or other apparatus. Any or all of the computer 102, the laptop 104 and the tablet device 118 can include engines. As used in this paper, an engine includes a dedicated or shared processor and, typically, firmware or software modules that the processor executes. Depending upon implementation-specific or other considerations, an engine can have a centralized distributed location and/or functionality. An engine can include special purpose hardware, firmware, or software embodied in a computer-readable medium for execution by the processor. A computer-readable medium is intended to include all mediums that are statutory (e.g., in the United States, under 35 U.S.C. 101), and to specifically exclude all mediums that are non-statutory in nature to the extent that the exclusion is necessary for a claim that includes the computer-readable medium to be valid. Known statutory computer-readable mediums include hardware (e.g., registers, random access memory (RAM), non-volatile (NV) storage, to name a few), but may or may not be limited to hardware. Any or all of the computer 102, the laptop 104 and the tablet device 118 can include one or more data-stores. A data-store can be implemented as software embodied in a physical computer-readable medium on a general- or specific-purpose machine, in firmware, in hardware and in a combination thereof, or in an applicable known or convenient device or system. Data-stores in this paper are intended to include any organization of data, including tables, comma-separated values (CSV) files, traditional databases (e.g., SQL), or other applicable known or convenient organizational formats. Data-store-associated components, such as database interfaces, can be considered “part of” a data-store, part of some other system component, or a combination thereof, though the physical location and other characteristics of data-store-associated components is not critical for an understanding of the techniques described in this paper. Data-stores can include data structures. A data structure is associated with a particular way of storing and organizing data in a computer so for efficient use within a given context. Data structures are based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The desktop computer 102, the laptop 104 or the tablet device 114 can function as network clients. Any or all of the desktop computer 102, the laptop 104 and the tablet device 114 can include one or more operating system software as well as application system software. The desktop computer 102, the laptop 104 or the tablet device 114 run a version of a Windows operating system from Microsoft Corporation, a version of a Mac operating system from Apple Corporation, a Linux based operating system such as an Android operating system, a Symbian operating system, a Blackberry operating system or other operating system. The desktop computer 102, the laptop and the tablet device 114 can also run one or more applications with which end-users can interact. The desktop computer 102, the laptop 104 and the tablet device 114 can run word processing applications, spreadsheet applications, imaging applications and other applications. Any or all of the desktop computer 102, the laptop 104 and the tablet device 114 can also run one or more programs that allow a user to access content over the network 108. Any or all of the desktop computer 102, the laptop 104 and the tablet device 114 can include one or more web browsers that access information over the network 108 by Hypertext Transfer Protocol (HTTP). The desktop computer 102, the laptop 104 and the tablet device 114 can also include applications that access content via File Transfer Protocols (FTP) or other standards. The desktop computer 102, the laptop 104 or the tablet device 114 can also function as servers. A server is an electronic device that includes one or more engines dedicated in whole or in part to serving the needs or requests of other programs and/or devices. The discussion of the servers 106, 110 and 112 provides further details of servers.

Referring to FIG. 2 in conjunction with FIG. 1 the desktop computer 102, the laptop 104 or the tablet device 114 can distribute data and/or processing functionality across the network 108 to facilitate providing cloud application imaging services. Any of the desktop computer 102, the laptop 104 and the tablet device 114 can incorporate modules such as the cloud-based server engine 200. Any of the server 106, the server 110 and the server 112 can include computer systems. Any of the server 106, the server 110 and the server 112 can include one or more engines. Any of the server 106, the server 110 and the server 112 can incorporate one or more data-stores. The engines in any of the server 106, the server 110 and the server 112 can be are dedicated in whole or in part to serving the needs or requests of other programs and/or devices. Any of the server 106, the server 110 and the server 112 can handle relatively high processing and/or memory volumes and relatively fast network connections and/or throughput. The server 106, the server 110 and the server 112 may or may not have device interfaces and/or graphical user interfaces (GUIs). Any of the server 106, the server 110 and the server 112 can meet or exceed high availability standards. The server 106, the server 110 and the server 112 can incorporate robust hardware, hardware redundancy, network clustering technology, or load balancing technologies to ensure availability. The server 106, the server 110 and the server 112 can incorporate administration engines that from electronic devices such as the desktop computer 102, the laptop computer 104 or the tablet device 114, or other devices access remotely through the network 108. Any of the server 106, the server 110 and the server 112 can include an operating system that is configured for server functionality, i.e., to provide services relating to the needs or requests of other programs and/or devices. The operating system in the server 106, the server 110 or the server 112 can include advanced or distributed backup capabilities, advanced or distributed automation modules and/or engines, disaster recovery modules, transparent transfer of information and/or data between various internal storage devices as well as across the network, and advanced system security with the ability to encrypt and protect information regarding data, items stored in memory, and resources. The server 106, the server 110 and the server 112 can incorporate a version of a Windows server operating system from Microsoft Corporation, a version of a Mac server operating system from Apple Corporation, a Linux based server operating system, a UNIX based server operating system, a Symbian server operating system, a Blackberry server operating system, or other operating system. The server 106, the server 110 and the server 112 can distribute functionality and/or data storage. The server 106, the server 110 and the server 112 can distribute the functionality of an application server and can therefore run different portions of one or more applications concurrently. Each of the server 106, the server 110 and the server 112 stores and/or executes distributed portions of application services, communication services, database services, web and/or network services, storage services, and/or other services. The server 106, the server 110 and the server 112 can distribute storage of different engines or portions of engines. For instance, any of the server 106, the server 110 and the server 112 can include some or all of the engines shown in the cloud-based server engine 200. The networking system 100 can include the network 108. The network 108 can include a networked system that includes several computer systems coupled, such as a local area network (LAN), the Internet, or some other networked system. The term “Internet” as used in this paper refers to a network of networks that uses certain protocols, such as the TCP/IP protocol, and possibly other protocols such as the HTTP for hypertext markup language (HTML) documents that make up the World Wide Web. Content servers, which are “on” the Internet, often provide the content. A web server, which is one type of content server, is typically at least one computer system, which operates as a server computer system, operates with the protocols of the World Wide Web, and has a connection to the Internet. Applicable known or convenient physical connections of the Internet and the protocols and communication procedures of the Internet and the web are and/or can be used. The network 108 can broadly include anything from a minimalist coupling of the components illustrated to every component of the Internet and networks coupled to the Internet. Components that are outside of the control of the networking system 100 are sources of data received in an applicable known or convenient manner. The network 108 can use wired or wireless technologies, alone or in combination, to connect the devices inside the networking system 100. Wired technologies connect devices using a physical cable such as an Ethernet cable, digital signal link lines (T1-T3 lines), or other network cable. The private network group 120 includes a wired local area network wired personal area network (PAN), a wired LAN, a wired metropolitan area network, or a wired wide area network. Some or all of the network 108 may include cables that facilitate transmission of electrical, optical, or other wired signals. Some or all of the network 108 can also employ wireless network technologies that use electromagnetic waves at frequencies such as radio frequencies (RF) or microwave frequencies. The network 108 includes transmitters, receivers, base stations, and other equipment that facilitates communication via electromagnetic waves. Some or all of the network may include a wireless personal area network (WPAN) technology, a wireless local area network (WLAN) technology, a wireless metropolitan area network technology, or a wireless wide area network technology. The network 108 can use Global System for Mobile Communications (GSM) technologies, personal communications service (PCS) technologies, third generation (3G) wireless network technologies, or fourth generation (4G) network technologies. The network 108 may also include all or portions of a Wireless Fidelity (Wi-Fi) network, a Worldwide Interoperability for Microwave Access (WiMAX) network, or other wireless network. The networking system 100 can include the private network group 120. The private network group 120 is a group of computers that form a subset of the larger network 108. The private network group 120 can include the laptop computer 122, the desktop computer 124, the scanner 126, a tablet device 128, the access gateway 62, the first physical device 64, the second physical device 66 and the third physical device 68. The laptop computer 122 can be similar to the laptop computer 104 the desktop computer 124 can be similar to the desktop computer 102, and the tablet device 128 can be similar to the tablet device 114. Any of the laptop computer 122, the desktop computer 124, the scanner 126, the tablet device 128, the access gateway 62, the first physical device 64, the second physical device 66 and the third physical device 68 can include computer systems, engines, data-stores. The private network group 120 can include a private network. A private network provides a set of private internet protocol (IP) addresses to each of its members while maintaining a connection to a larger network, here the network 108. To this end, the members of the private network group 120 (i.e., the laptop computer 122, the desktop computer 124, the scanner 126, the tablet device 128, the first physical device 64, the second physical device 66 and the third physical device 68) can each be assigned a private IP address irrespective of the public IP address of the router 62. Though the term “private” appears in conjunction with the name of the private network group 120 the private network group 120 includes a public network that forms a subset of the network 108. In such a case, each of the laptop computer 122, the desktop computer 124, the scanner 126, the tablet device 128, the first physical device 64, the second physical device 66 and the third physical device 68 can have a public IP address and can maintain a connection to the network 120. The connection of some or all of the laptop computer 122, the desktop computer 124, the scanner 126, the tablet device 128, the first physical device 64, the second physical device 66 and the third physical device can be a wired or a wireless connection. The private network group 120 includes the access gateway 62. The access gateway 62 assigns private IP addresses to each of the devices 122, 124, 126, 128, 64, 66 and 68. The access gateway 62 can establish user accounts for each of the devices 122, 124, 126, 128, 64, 66 and 68 and can restrict access to the network 108 based on parameters of those user accounts. The access gateway 62 can also function as an intermediary to provide content from the network 108 to the devices 122, 124, 126, 128, 64, 66 and 68. The access gateway 62 can format and appropriately forward data packets traveling over the network 108 to and from the devices 122, 124, 126, 128, 64, 66 and 68. The access gateway 62 can be a router, a bridge, or other access device. The access gateway 62 can maintain a firewall to control communications coming into the private network group 120 through the network 108. The access gateway 62 can also control public IP addresses associated with each of the laptop computer 122, the desktop computer 124, the scanner 126, the tablet device 128, the first physical device 64, the second physical device 66 and the third physical device 68. The access gateway 62 is absent and each of the devices inside the private network group 120 can maintain its own connection to the network 108. The desktop computer 124 is shown connected to the access gateway 62 as such a configuration is a common implementation. The functions described in relation to the desktop computer 124 can be implemented on the laptop computer 122, the tablet device 128, or any applicable computing device. The private network group 120 can be located inside a common geographical area or region. The private network group 120 can be located in a school, a residence, a business, a campus, or other location. The private network group 120 is located inside a health office, such as the office of a dentist, a doctor, a chiropractor, a psychologist, a veterinarian, a dietician, a wellness specialist, or other health professional. The physical devices 64, 66 and 68 can image a physical object. The physical devices 64, 66 and 68 can connect to the desktop computer 124 via a network connection or an output port of the desktop computer 124. Similarly, the physical devices 64, 66 and 68 can connect to the laptop computer 122, the tablet device 128, or a mobile phone. The physical devices 64, 66 and 68 are directly connected to the access gateway 62. The physical devices 64, 66 and 68 can also internally incorporate network adapters that allow a direct connection to the network 108. The first physical device 64 can be a sensor-based imaging technology. A sensor is a device with electronic, mechanical, or other components that measures a quantity from the physical world and translates the quantity into a data structure or signal that a computer, machine, or other instrument can read. The first physical device 64 can use a sensor to sense an attribute of a physical object. The physical object can include, for instance, portions of a person's mouth, head, neck, limb, or other body part. The physical object can be an animate or inanimate item. The sensor may include x-ray sensors to determine the boundaries of uniformly or non-uniformly composed material such as part of the human body. The sensor can be part of a Flat Panel Detector (FPD). Such an FPD can be an indirect FPD including amorphous silicon or other similar material used along with a scintillator. The indirect FPD can allow the conversion of X-ray energy to light, which is eventually translated into a digital signal. Thin Film Transistors (TFTs) or Charge Coupled Devices (CCDs) can subsequently allow imaging of the converted signal. Such an FPD can also be a direct FPD that uses Amorphous Selenium or other similar material. The direct FPD can allow for the direct conversion of x-ray photons to charge patterns that, in turn, are converted to images by an array such as a TFT array, an Active Matrix Array, or by Electrometer Probes and/or Micro-plasma Line Addressing. The sensor may also include a High Density Line Scan Solid State detector. The sensor of the first physical device 64 may include an oral sensor. An oral sensor is a sensor that a user such as a health practitioner can insert into a patient's mouth. The first physical device 64 can reside in a dentist's office that operates the private network group 120. The sensor of the first physical device 64 may also include a sensor that is inserted into a person's ear, nose, throat or other part of a person's body. The second physical device 66 may include a digital radiography device. Radiography uses x-rays to view the boundaries of uniformly or non-uniformly composed material such as part of the human body. Digital radiography is the performance of radiography without the requirements of chemical processing or physical media. Digital radiography allows for the easy conversion of an image to a digital format. The digital radiography device can be located in the office of a health professional. The third physical device 68 may include a thermal-based imaging technology. Thermal imaging technology is technology that detects the presence of radiation the infrared ranges of the electromagnetic spectrum. Thermal imaging technology allows the imaging of the amount of thermal radiation emitted by an object. The third physical device 68 may include an oral sensor, or a sensor that is inserted into a person's ear, nose, throat, or other part of a person's body. The third physical device 68 resides in the office of a health professional, such as the office of a dentist, a doctor, a chiropractor, a psychologist, a veterinarian, a dietician, a wellness specialist or other health professional. The networking system 100 can facilitate delivery of a cloud application imaging service. A cloud application imaging service is a service that allows an entity associated with a physical device (such as one of the physical devices 64, 66 and 68) to use a cloud-computing application that is executed on a client computer (such as the desktop computer 124) to direct the physical device to image a physical object. Cloud-based computing, or cloud computing, is a computing architecture in which a client can execute the full capabilities of an application in a container (such as a web browser). Though the application executes on the client, portions of the application can be distributed at various locations across the network. Portions of the cloud application imaging service that are facilitated by the networking system 100 can reside on one or more of the desktop computer 102, the laptop computer 104, the server 106, the server 110 the server 112, the tablet device 114, and/or other locations “in the cloud” of the networking system 100. The application can appear as a single point of access for an end-user using a client device such as the desktop computer 124. The cloud application imaging service can implement cloud client functionalities onto the desktop computer 124. A cloud client incorporates hardware and/or software that allows a cloud application to run in a container such as a web browser. Allowing the desktop computer 124 to function as a cloud client requires the presence of a container in which the cloud application imaging service can execute on the desktop computer 124. The cloud application imaging service can facilitate communication over a cloud application layer between the client engines on the desktop computer 124 and the one or more server engines on the desktop computer 102, the laptop computer 104, the server 106, the server 110 the server 112, the tablet device 114, and/or other locations “in the cloud” of the networking system 100. The cloud application layer or “Software as a Service” (SaaS) facilitates the transfer over the Internet of software as a service that a container, such as a web browser, can access. Thus, as discussed above, the desktop computer 124 need not install the cloud application imaging service even though the cloud application imaging service executes on the desktop computer 124. The cloud application imaging service can also deliver to the desktop computer 124 one or more Cloud Platform as a Service (PaaS) platforms that provide computing platforms, solution stacks, and other similar hardware and software platforms. The cloud application imaging service can deliver cloud infrastructure services, such as Infrastructure as a Service (IaaS) that can virtualize and/or emulate various platforms, provide storage, and provide networking capabilities. The cloud application imaging service, consistent with cloud-computing services in general, allows users of the desktop computer 124 to subscribe to specific resources that are desirable for imaging and other tasks related to the physical devices 64, 66 and 68. Providers of the cloud application imaging service can bill end-users on a utility computing basis, and can bill for use of resources. In the health context, providers of the cloud application imaging service can bill for items such as the number of images an office wishes to process, specific image filters that an office wishes to use, and other use-related factors.

Referring to FIG. 2 in conjunction with FIG. 1 either part or all of the cloud application imaging service can reside on one or more server engines. A conceptual diagram of a cloud-based server engine 200 includes a device search engine 202 that searches the physical devices connected to a client computer. The cloud-based server engine 200 may also include remote storage 204 that includes one or more data-stores and/or memory units. The remote storage 204 can include storage on Apache-based servers that are available on a cloud platform such as the EC2 cloud platform made available by Amazon. The cloud-based server engine 200 can may include a physical device selection engine 206 that selects a specific physical device connected to a client. The cloud-based server engine 200 can include a physical device configuration engine 208 that configures image parameters and/or attributes of the specific physical device. An image selection engine 210 inside the cloud-based server engine 200 can allow the selection of a specific image from the physical device. A communication engine 212 inside the cloud-based server engine 200 allow the transfer of selection data, parameter data, device data, image data, and other data over a network such as the network 108. The cloud-based server engine 200 includes a content engine 214 that makes images available to client devices associated with a cloud application imaging service. Processors can control any or all of the components of the cloud-based server engine 200 and these components can interface with data-stores. Any or all of the cloud-based server engine 200 can reside on a computing device such as the desktop computer 102, the laptop 104, the tablet device 114, the server 106, the server 110 and/or server 112. Portions of the cloud-based server engine 200 can also be distributed across multiple electronic devices, including multiple servers and computers.

Referring to FIG. 3 in conjunction with FIG. 1 a cloud-based client system 300 includes the network 108, the first physical device 64, the second physical device 66 and the third physical device 68. Each of the network 108, the first physical device 64, the second physical device 66 and the third physical device 68. The cloud-based client system 300 also includes a cloud-based media acquisition client 304 which can reside inside a computer, such as the desktop computer 124. The cloud-based media acquisition client 304 also interfaces with the network 108. The access gateway 62 allows the cloud-based media acquisition client 304 to communicate with the network 108. The cloud-based media acquisition client 304 can also be connected to the network 108 through other I/O devices and/or means. The cloud-based media acquisition client 304 is also connected to the first physical device 64, the second physical device 66 and the third physical device 68. Either a network connection or an I/O device and/or means can facilitate the connections between the cloud-based media acquisition client 304 and any of the first physical device 64, the second physical device 66 and the third physical device 68.

U.S. Patent Application Publication No. 2011/0304740 teaches a universal image capture manager (UICM) which facilitates the acquisition of image data from a plurality of image source devices (ISDs) to an image utilizing software (IUSA). The universal image capture manager is implemented on a computer processing device and includes a first software communication interface configured to facilitate data communication between the universal image capture manager and the image utilizing software. The universal image capture manager also includes a translator/mapper (T/M) software component being in operative communication with the first software communication interface and configured to translate and map an image request from the image utilizing software to at least one device driver software component of a plurality of device driver software components. The universal image capture manager further includes a plurality of device driver software components being in operative communication with the translator/mapper software component. Each device driver software components is configured to facilitate data communication with at least one image source device. Many times it is desirable to bring images into a user software. This is often done in the context of a medical office environment or a hospital environment. Images may be captured by image source devices such as a digital camera device or an x-ray imaging device and are brought into a user software such as an imaging software or a practice management software running on either a personal computer or a workstation. Each image source device may require a different interface and image data format for acquiring image data from that image source device. The various interfaces may be TWAIN-compatible or not, may be in the form of an application program interface (API), a dynamic link library (DLL) or some other type of interface. The various image data may be raw image data, DICOM image data, 9-bit or 32-bit or 64-bit image data, or some other type of image data. The process of acquiring an image into a user software can be difficult and cumbersome. In order to acquire and place an image in a user software a user may have to first leave the software, open a hardware driver, set the device options, acquire the image, save the image to a local storage area, close the hardware driver, return to the software, locate the saved image, and read the image file from the local storage area. Hardware and software developers have developed proprietary interfaces to help solve this problem. Having a large number of proprietary interfaces has resulted in software developers having to write a driver for each different device to be supported. This has also resulted in hardware device manufacturers having to write a different driver for each software. General interoperability between user software and image source devices has been almost non-existent. The imaging modality may be an intra-oral x-ray modality, a pan-oral x-ray modality, an intra-oral visible light camera modality, or any other type of imaging modality associated with the system. The anatomy may be one or more teeth numbers, a full skull, or any other type of anatomy associated with the system. The operatory may be operatory #1, or operatory #4, a pan-oral operatory, an ultrasound operatory, or any other type of operatory associated with the system. The work-list may be a work0-list from a Picture Archiving and Communication System (PACS) server where the work-list includes a patient name. The specific hardware type may be a particular type of intra-oral sensor or a particular type of intra-oral camera. The patient type may be pediatric, geriatric, or adolescent. The interface is configured to access the clipboard of a computer processing device and paste the returned image data set to the clipboard. The universal image capture manager may be configured to enable all of the plurality of device drivers upon receipt of an image request message and, if any image source device of the plurality of image source devices has newly acquired image data to return, the newly acquired image data will be automatically returned to the image utilizing software through the universal image capture manager.

Referring to FIG. 4 a system 400 includes an image utilizing software (IUSA) 410 which is implemented on a first computer processing device 411, a universal image capture manager (universal image capture manager) 420 which is implemented on a second computer processing device 421 and a plurality of image source devices (ISDs) 430 (e.g., ISD #1 to ISD #N, where N represents a positive integer) in order to acquire image data from multiple sources. The image utilizing software 410 may be a client software such as an imaging software or a practice management application as may be used in a physician's office, a dentist's office, or a hospital environment. The image utilizing software 410 is implemented on the first computer processing device 411, such as a personal computer (PC) or a work station computer. There is a plurality of image source devices 430 which are hardware-based devices that are capable of capturing images in the form of image data (e.g., digital image data). Such image source devices 430 include a visible light intra-oral camera, an intraoral x-ray sensor, a panoramic (pan) x-ray machine, a cephalometric x-ray machine, a scanner for scanning photosensitive imaging plates and a digital endoscope. There exist many types of image source devices using many different types of interfaces and protocols to export the image data from the image source devices. The universal image capture manager 420 is a software or a software module. The second computer processing device 421, having the universal image capture manager 420, operatively interfaces between the first computer processing device 411, having the image utilizing software 410 and the plurality of image source devices 430, and acts as an intermediary between the image utilizing software 410 and the plurality of image source devices 430. The universal image capture manager 420 is a software module implemented on the second computer processing device 421 such as a personal computer, a workstation computer, a server computer, or a dedicated processing device designed specifically for universal image capture manager operation. The universal image capture manager 420 is configured to communicate in a single predefined manner with the image utilizing software 410 to receive image request messages from the image utilizing software image utilizing software 410 and to return image data to the image utilizing software 410. The universal image capture manager 420 is configured to acquire image data from the multiple image source devices 430. As a result, the image utilizing software 410 does not have to be concerned with being able to directly acquire image data from multiple different image data sources itself. Instead, the universal image capture manager 420 takes on the burden of communicating with the various image source devices 430 with their various communication interfaces and protocols.

Referring to FIG. 5 in conjunction with FIG. 4 a universal image capture manager 420 (UICM) software module architecture used in the system 400 includes a first software interface that is a universal image capture manager/image utilizing software interface 510 that is configured to facilitate data communication between the universal image capture manager 420 and the image utilizing software 410. The interface 510 may be a USB interface, an Ethernet interface, or a proprietary direct connect interface. The interface 510 is implemented in software and operates with the hardware of the second computer processing device 421 to input and output data (e.g., image request message data and image data) from/to the image utilizing software 410. The universal image capture manager 420 further includes a plurality of device drivers 530 (e.g., DD #1 to device driver DD #N, where N is a positive integer). The device drivers 530 are implemented as software components and operate with the hardware of the second computer processing device 421 to input and output data (e.g., image data and device driver access data) from/to the plurality of image source devices 430. Each device driver 530 is configured to facilitate data communication with at least one of the image source devices 430. A device driver of the plurality of device drivers 530 may be a TWAIN-compatible device driver provided by a manufacturer of at least one corresponding image source device 430. TWAIN is a well-known standard software protocol that regulates communication between software and image source devices 430. TWAIN is not an official acronym but is widely known as “Technology without an Interesting Name.” Another device driver 530 may be a TWAIN-compatible or a non-TWAIN-compatible direct driver interface developed using a software development kit (SDK) provided by a manufacturer of at least one corresponding image source device 430. The software development kit includes a compiler, libraries, documentation, a code, an integrated development environment and a simulator for testing the code. A device driver 530 may be a custom application programming interface (API). The application programming interface is an interface implemented by a software program which enables interaction with other software program. A device driver 530 may be part of a dynamic link library (DLL). The dynamic link library is a library that contains code and data that may be used by more than one software program at the same time and promotes code reuse and efficient memory usage. The universal image capture manager 420 is configured to be able to readily either add or remove a device driver software component to the plurality of device drivers 530. The universal image capture manager 420 is configured in a software “plug-n-play” manner so that device drivers may be readily either add device drivers or remove them without having to reconfigure any of the other device drivers. The universal image capture manager 420 may be easily adapted as image source devices 430 of the system 400 are able to add device drivers, change them out, upgraded them, replace them or discard them. The universal image capture manager 420 also includes a translator/mapper (T/M) 520 software component. The translator/mapper 520 is in operative communication with the universal image capture manager/image utilizing software interface 510 and the plurality of device drivers 530. The translator/mapper 520 is configured to translate and map an image request from the image utilizing software 410 to at least one device driver of the plurality of device drivers 530. The translator/mapper 520 is configured to translate and map image data received from at least one image source device of the plurality of image source devices 430 via at least one device driver of the plurality of device drivers 530. The computer-executable software instructions of the universal image capture manager 420 may be stored on a non-transitory computer-readable medium. The non-transitory computer-readable medium may include a compact disk (CDROM), a digital versatile disk (DVD), a hard drive, a flash memory, an optical disk drive, random access memory (RAM), read only memory (ROM), electronically erasable programmable read only memory (EEPROM), magnetic storage devices such as magnetic tape or magnetic disk storage, or any other medium that can be used to encode information that may be accessed by a computer processing device.

U.S. Patent Application Publication No. 2017/0168812 teaches a method for integrating a non-supported dental imaging device into dental imaging software which operates on a computer. The computer is coupled to a display that is capable of displaying dental x-rays and dental photographs. An originally supported dental imaging device has an API binary file with an original filename accessible to the computer. The method includes the steps of creating a replacement alternate API binary file which contains equivalent functionality as the API binary file of the original supported dental imaging device and placing the replacement alternate API binary file either onto or accessible to the computer. The replacement alternate API binary file has the same filename as does the original filename of the API binary file of the originally supported dental imaging device. The method also includes the step of having the replacement alternate API binary file operated on by the dental imaging software by means of the computer. The dental imaging software is not aware the dental imaging software is not communicating with the originally supported dental imaging device. The replacement alternate API binary file delivers image data acquired by the non-supported imaging device to the dental imaging software.

Referring to FIG. 6 a dental office 600 includes a computer 610 and a display 611. The computer 610 includes a microprocessor 612, a memory 613, such as a random access memory (RAM), and a non-volatile storage or memory 614, such as either a hard disk or a flash memory, for storing software or data. The computer 610 may be coupled either directly or indirectly to the display 611. The display 611 is capable of displaying dental images including dental x-rays and dental photographs. The computer 610 has an operating system 615 which may be either a Windows based operating system or a Mac OS X based operating system or another compatible operating system. The computer 610 may also be a mobile computer, such as an iPad, an Android based tablet, a Microsoft Surface based tablet, a phone, or any other proprietary device with an adequate microprocessor, an operating system and a display which is capable of displaying dental images including dental x-rays and dental photographs.

Still referring to FIG. 6 the dental office 600 also includes dental imaging software 620 having a sub-section 630 which integrates and acquires images from a specific supported or proprietary imaging device using the API binary file 640 of the specifically supported proprietary imaging device. The dental imaging software 620 is either legacy dental imaging software or proprietary dental imaging software. The first imaging device 650 is a specifically originally supported native imaging device. The second imaging device 660 is also a specifically supported native imaging device. The first imaging device 650 may be either a 2D intraoral or a 2D extraoral dental imaging device. The second imaging device 660 may be either a 3D intraoral or a 3D extraoral dental imaging device. The group of supported dental imaging devices may consist of 2D intraoral x-ray sensors, 3D intraoral x-ray sensors, 2D extraoral x-ray sensors, 3D extraoral x-ray sensors, dental camera, dental image data sources, dental imaging acquisition devices, dental images stored in a non-volatile memory such as either a hard disk drive or a flash drive, imaging plate scanner sensors and any other diverse dental image sources.

Referring still further to FIG. 6 the dental office 600 does not use the claimed invention. The computer 610 operates imaging software 620. The dental imaging software 620 may be either running locally on the computer 610 or displaying the results of software operating upon a remote server, such as either web-based dental imaging software or cloud-based dental imaging software. The imaging software 620 may be either directly controlling or indirectly controlling the first imaging device 650 using sub-section 630 of the dental imaging software 620. The dental imaging software 620 may also be either directly or indirectly controlling the second imaging device 660 using sub-section 630 of the dental imaging software 620. The sub-section 630 communicates with the API binary file 640 which in turn communicates with at least one of the first and second imaging devices 650 and 660 to direct imaging or receive images. The API binary file 640 is stored in either the non-volatile storage 614, or the memory 613 on the computer 610 or in another either non-volatile storage or memory either coupled to the computer 610 or accessible by computer 610. The imaging software 620 communicates to the sub-section 630 for the purpose of controlling the actions of at least one of the first and second imaging devices 650 or 660 using the API binary file 640 thereof. The sub-section 630 receives communication or status from the specific imaging device by means of its API binary file 640 which communicates directly or indirectly with the device driver of one of the first and second imaging devices 650 and 660. The communications between the imaging software 620 and sub-section 630 and to binary imaging device API binary file 640 are proprietary in nature. The API binary files are not universal for imaging devices and no two imaging devices typically have the same functions, parameters, or overall operation in their API binary file for that specific imaging device. Dental imaging software 620 commands the computer 610 to initiate and/or receive image or image data from either the first imaging device 650 or the second imaging device 660 by means of communication through sub-section 630 and its API binary file 640. After either an image or image data has been enacted by API binary file 640 it is made available to the dental imaging software 620 by means of the sub-section 630 or other means for any additional processing, storage and ultimately display upon computer 610.

Referring to FIG. 7 a dental office 700 includes a computer 710 and a display 711. The computer 710 includes a microprocessor 712, a memory 713, such as a random access memory (RAM), and a non-volatile storage or memory 714, such as either a hard disk or a flash memory, for storing software or data. The computer 710 may be coupled either directly or indirectly to the display 711. The display 711 is capable of displaying dental images including dental x-rays and dental photographs. The computer 710 has an operating system 715 which may be either a Windows based operating system or a Mac OS X based operating system, or another compatible operating system.

Referring still to FIG. 7 the dental office 700 uses the claimed invention. The computer 710 operates imaging software 720. The computer 710 may also be a mobile computer, such as an iPad, an Android based tablet, a Microsoft Surface based tablet, a phone or any other proprietary device with an adequate microprocessor, operating system and display capability. The dental office 700 also includes imaging software 720 having a sub-section 730 which integrates and acquires images from a specific or proprietary imaging device using the API binary file 740 of the specific or proprietary imaging device. The first imaging device 760 is an originally unsupported 2D imaging device. The second imaging device 770 is also an originally unsupported 3D imaging device. The first imaging device 760 may be a 2D intraoral or extraoral dental imaging device. The second imaging device 770 may be a 3D intraoral or extraoral dental imaging device. Originally supported dental imaging devices may consist of 2D intraoral x-ray sensors, 3D intraoral x-ray sensors, 2D extraoral x-ray sensors, 3D extraoral x-ray sensors, dental camera, dental image data sources, dental imaging acquisition devices, dental images stored in a non-volatile memory such as either a hard disk drive or a flash drive, imaging plate scanner sensors, PSP devices and any other diverse dental image sources. Originally non-supported dental imaging devices that become supported using the claimed invention may consist of 2D intraoral x-ray sensors, 3D intraoral x-ray sensors, 2D extraoral x-ray sensors, 3D extraoral x-ray sensors, dental camera, dental image data sources, dental imaging acquisition devices, dental images stored in a non-volatile memory such as either a hard disk drive or a flash drive, imaging plate scanner sensors, PSP devices and any other diverse dental image sources.

Referring still further to FIG. 7 the imaging software 720 may be either running locally on the computer 710 or displaying the results of software operating upon a remote server, such as web/cloud based imaging software. The imaging software 720 is communicating and/or controlling an originally/natively supported 2D intraoral or extraoral imaging device 780 and/or an originally supported 3D intraoral or extraoral imaging device 790. Sub-section 730 of the imaging software includes integration to a specific proprietary imaging device API binary file 740. The original specific imaging device API binary file 740 that sub-section 730 communicated with has been renamed to a different filename on computer 710 or on another device accessible to computer 710. The renamed original API binary 750 is accessible to replacement API binary file 740. The filename of replacement binary API file 740 is named the same as the original specific proprietary imaging device API binary filename and contains identical or near-identical functions as original API binary which are called by the decoupled imaging software to support the specific imaging device natively. The specific imaging device is a previously supported 2D intraoral or extraoral dental imaging device 780 or 790 3D imaging device and/or the imaging device is a previously unsupported 2D imaging device 760 or 3D intraoral or extraoral dental imaging device 770.

Referring yet still further to FIG. 7 the subsection 730 of the imaging software 720 includes a replacement API binary file 740 and shows the dental imaging software sub-section 730 communicating with the replacement API binary and controlling either the original natively supported imaging devices 780 and/or 790 or the non-natively supported imaging devices 760 and/or 770. The imaging software is unaware that it is not communicating with the original natively supported imaging device API binary as the function/parameters called and values returned are identical to the original natively supported API binary file in the replacement API binary file 740. When the imaging software sub-section 730 communicates with replacement API binary 740, the said API binary file 740 communicates with the renamed original API binary file 750 and relays the same functions and parameters as were communicated to it by means of the imaging software 720 and its sub-section 730; and which allows the original natively supported imaging devices 780 and 790 to continue to be supported in imaging software 720. Replaced binary API file 740 also communicates with non-supported imaging device 760 and 770 API/devices. Replacement API 740 translates, forwards, adds and deletes functions or parameters received from the imaging software to be compatible with the previously non-supported imaging device 760 or 770 and their API. Replaced binary API file 740 also translates or converts imaging device 760 or 770's API return codes, messages, or image data to be compatible with what sub-section 730 of imaging software 720 expects for functions, return values, and messages from the API binary file 740. The imaging software subsection 730 calls the same functions and/or parameters and/or methods for the originally natively supported device via the replacement API binary 740 which has the same functions as the original renamed API binary 750. The imaging software is not aware of any changes or that it is not communicating with the natively supported imaging devices via the original API binary file or files. The existing natively supported imaging devices 780 and 790 continue to operate and non-natively supported devices 760 and 770 can now operate within the decoupled imaging software. This is one hundred percent (100%) transparent to the imaging software so that no changes are required to the legacy or proprietary imaging software application.

Referring to FIG. 8 in conjunction with FIG. 7 a computer-implemented method for integrating a non-supported dental imaging device into dental imaging software operates on the computer 710 coupled to the display 711 which is capable of displaying dental x-rays and dental photographs. An originally supported dental imaging device has either an API binary file or API binary files with either an original filename or filenames, respectively, either directly or indirectly accessible to the computer 710. The computer-implemented method 800 includes the steps of operating a legacy or proprietary dental imaging software application which controls acquisition from a 2D or 3D imaging device upon a computing device. In step 810 the proprietary or legacy imaging software has been programmed to support specific 2D and/or 3D imaging devices using proprietary API's, and where the imaging software is configured to acquire images from one or more of the supported imaging devices. In step 820 the original binary API file or files for an originally supported device has been renamed to another filename. In step 830 a replacement API file with the same filename as the original API filename has been created and placed onto or accessible to the computing device. In step 830 the replacement API file is enacted upon by the imaging software to acquire images and is not aware it is not communicating with the original supported device API. In step 840 communication is received or initiated between the imaging software and the replacement binary API. In step 850 any messages sent or received from devices or the legacy application are arbitrated to the proper proprietary API/device. In step 860 any communicated between the API and the imaging software are translated, converted transparently to the imaging application software. In step 870 the image or image data is delivered from the previously supported or un-supported device transparently in that the imaging software does not know it is not receiving images or communication from the originally supported device and device API. Thereby allowing support for a specific previously unsupported dental imaging device by the dental imaging software while the dental imaging software is configured to support an originally supported imaging device. The computer-implemented method may include either the step of renaming the original filename or filenames of either the API binary file or the API binary files of the originally supported dental imaging device on the computer 710 or the step of deleting either the original filename or the original filenames of either the API binary file or the API binary files of the originally supported dental imaging device on the computer 710. An alternate application programming interface (API) may control two or more connected dental imaging devices simultaneously. The computer-implemented method includes a non-transitory computer-readable medium storing computer-executable application programming interface (API) for use with the computer. The non-transitory computer-readable medium includes a set of instructions which allows integration of the non-supported imaging devices into dental imaging software.

U.S. Pat. No. 6,041,362 teaches a method for integrating disparate information technology applications and platforms across an enterprise which provides a web client interface that associates with an enterprise network. Connecting with the web client through the network is the Hyper-Text Transfer Protocol (HTTP) server that includes a Common Gateway Interface (CGI) interface program for augmenting the integration of the disparate applications and platforms via remote and local applications execution. The HTTP server is specific to the particular enterprise for specifically dealing with application servers and information servers and further for collecting information and gathering it together into a form that is then displayed on the web client. The qeb client interface connects through an enterprise network to an application integrating server such as a Hyper-Text Transfer Protocol (HTTP) server. The HTTP server includes a graphical interface such as that provided by a Common Gateway Interface (CGI) interface program for integrating the disparate applications and platforms of the enterprise via remote and local applications execution. The HTTP server may be specific to the particular enterprise for specifically integrating and interfacing with application servers and information servers and further for collecting and gathering information together to display it in a desired form on the CGI interface. The HTTP server may include a form that is displayed in a Hyper-Text Markup Language (HTML) format. The user provides information that the form requires, and, in response, the HTTP server causes the execution of CGI script that may contain the logic and other instructions for sending a request that executes a transaction to an addressed and interfaced application server. The application server then may respond to the request, update information relating to that user's request by addressing the particular application server, and providing the results of the initiated activity. The HTTP server then can build a new or supplemental existing HTML document that the HTTP server returns to the web client. The user may then respond, as desired, to the built document by seeking more or different information from the different applications and platforms of the enterprise. This architecture includes the steps of identifying all the components to make this work. The enterprise network is specific to the particular enterprise according to the various disparate applications and platforms within the enterprise. The HTTP server includes a CGI interface program that augments integration via remote and local application execution. The CGI interface is unique to the particular enterprise and specifically deals with the particular application servers or information servers of the enterprise to collect the information, gather it together, and assemble it in a form that the user interface displays. The CGI script contains the logic and instructions for sending a request for a transaction to an appropriate application server and for receiving a response back from that application server. The CGI interface program may be written in C, PERL, or some other appropriate computer language that permits the formation of logic and instructions for the particular application server.

Referring to FIG. 9 an enterprise network 910 interfaces with client workstation 912 that operates on the web client. The where the web client includes a computer software application. The client workstation 912 is used by a user or operator, and the web client is run by client workstation 912 for use by the user or operator. A HTTP server 914 includes a CGI interface program that augments integration via a remote and local application execution and connects with the enterprise network cloud 910. Application servers 916 connects to enterprise network cloud 910 and includes numerous and disparate application servers, such as an application server for member database 918, an application server for employee file 920, and an application server for order file and program 922. Also connecting to enterprise network cloud 910, information servers 924 may include numerous and disparate information servers, such as information servers for an organization charge data file 926, a member handbook 928, and a remote application library 930. The enterprise network cloud 910 also connects or has an interface with HTTP server 932. The integrated application includes a disparate application across an enterprise. At step 1-1, the user at client workstation 912 fills in the HTML form to request information relating to membership of an individual and submits the form by clicking a SUBMIT button or function key at client workstation 912. Step 1-2 shows that the HTML form is received by HTTP server 932 and an Add Member function of the CGI interface program indicated in the HTML form is to be executed. At step 1-3, the Add Member function of the CGI interface program connects to application servers 916 and the Add Member Transaction is initiated by application server 916. The Add Member Transaction, at step 1-4, queries the employee database of employee file 920 to validate and edit the information from the employee database in response to the request. At step 1-5, the Add Member Transaction ensures that the employee is not currently a member and then adds the employee to membership database 918. Code indicating a successful transaction is returned at step 1-6 to the Add Member CGI program currently executing on the HTTP Server. The Add Member CGI program, at step 1-7, sends a request to Information Servers 924 updating member handbook 928 to include the new member in its member directory. Member handbook 928 is an online file that may be in a word processing format. At step 1-8, member handbook 928 is updated by an Update Member Handbook Application through information server 924. The HTML Version of the member directory, which may reside on yet another HTTP Server is also updated at step 1-9, by the update member handbook application. There are at least two ways to update member handbook 928. One way is to have the server access the database directly and return the results. One of the many possible alternatives to the above method to update addresses the situation of HTTP server 932 not having the ability or clearance to go to member database 918, for one reason or another. An alternative approach, in this instance, would be to build an HTML tree that contains the entire member database 918. At step 1-10, code indicating a successful transaction is returned to the add member CGI program along with the specification of the URL for the current Member Handbook. This results in the construction of an HTML page that indicates the successful status of the request and contains a hotlink to the new Member Handbook. The resulting HTML page is then sent, at step 1-11, to client workstation 912 where it is displayed. The display notifies the user that his application was successful and gives him a hot link to the updated Member Handbook 928. The user can then initiate viewing of the new Member Handbook by pressing the associated hotlink.

U.S. Patent Application Publication No. 2014/0143298 teaches a system for zero footprint medical image-viewing which includes a zero-footprint viewer including a display pipeline to render and provide image content to a client device without particular configuration of the client device to display and facilitate manipulation of the image content via a client browser. The system also includes a middle-tier server to retrieve the image content from storage and to convert the image content from a stored format to a browser-convenient format. The zero footprint viewer includes a first data manager to gather image content from the middle-tier server, and the middle-tier server includes a second data manager to retrieve the image content and format the image content from the stored format to the browser-convenient format, the second data manager to communicate with the first data manager to facilitate transfer of the image content for display. Prior to the rapid onset of digital imaging, patient images were “printed” to film. The film was “hung” and viewed by radiologists, who would then dictate a report. Reports were transcribed by individuals ranging for administrative staff to medical transcriptionists and sent to ordering physician via mail or fax. Critical results were delivered by phone or pager and business statistics were managed via paper reports and spreadsheets. As information systems for radiology came to market, the first commercially available solutions addressed the needs of the radiologist and the radiology department. These included Radiology Information Systems (RIS) and dictation transcription systems. RIS systems managed the ordering, scheduling, patient and management reporting processes while radiologists were still reading from film. As modalities started to support the digital display of images on workstations connected to the acquisition device, Picture Archiving and Communications Systems (PACS) came to market. These centrally store images and provide radiologists with the tools to read studies on networked computer monitors, replacing both film and modality workstations. Over time, the needs of the market have evolved from supporting specialized radiologist workflows to supporting the open and dynamic needs of the enterprise and the community. The vendor community has added systems to manage the need for advanced technologies for better diagnosis; the sharing of images between providers and organizations; to support collaboration between radiologists, physicians and teams providing care for the patient; to close the loop on reporting of critical results and manage the growing storage requirements. Often these are disparate, best-of breed systems that may or may not interoperate, increasing cost and decreasing productivity.

U.S. Patent Application Publication No. 2012/0253848 teaches a system which integrates different applications like imaging system, audio-video streaming system, Electronic Medical Records (EMR), Electronic Health Records (EHR), Patient Health Records (PHR), PACS (Picture Archiving and Communication System), Lab System and patient monitoring systems, databases or warehouses into single application, wherein all the applications are displayed in a single computer screen/interface preferably by using a common interaction platform such as a web browser. The web browser provides the patient direct interface in real time with the health care professional. The system enables standardized data, records and content of patients, store the information captured into integrated application database and/or into its objects stored in the application folders. The system integrates different applications like image archiving system, audio-video streaming system, Electronic Medical Records (EMR), Electronic Health Records (EHR), Patient and Health Records (PHR), PACS (Picture Archiving and Communication System), Lab System and patient monitoring systems/databases/warehouses into single application, wherein all the applications are displayed in a single screen/interface to read write to these systems/applications if authorized. The platform provides integrated application interface by way of a web browser in a single computer screen. Healthcare environments, such as hospitals or clinics, include clinical information systems, such as hospital information systems (HIS), radiology information systems (RIS), clinical information systems (CIS), and cardiovascular information systems (CVIS), and storage systems, such as picture archiving and communication systems (PACS), library information systems (LIS), and Electronic Medical Records (EMR) or Electronic Health Records (EHR). Information stored may include patient medical histories, imaging data, test results, diagnosis information, management information, and/or scheduling information. The information may be centrally stored or divided among a plurality of locations. Healthcare practitioners may desire to access patient information or other information at various points in a healthcare workflow. During surgery, medical personnel may access patient information, such as images of a patient's anatomy, which are stored in a medical information system. Alternatively, medical personnel may enter new information, such as history, diagnostics, or treatment information, into a medical information system during an ongoing medical procedure. In a healthcare workflow, healthcare providers often consult or otherwise interact with each other. Such interaction typically involves paging or telephoning another practitioner. Interaction between healthcare practitioners may be both time-consuming and energy-consuming. Current advances in technologies related to medical/healthcare delivery services use one or more applications to review a patient history, medical information and record information during interactions. The state of the applications today is disparate and requires un-friendly actions by the users to obtain information. To review images within an imaging system (e.g. PACS—Picture Archiving and Communication System) while reviewing/updating medical records in an electronic medical records (EMR), the medical user will have to close EMR, login into PACS, review images, print/hand write information, close PACS, re-login into EMR and enter the collected information. Current healthcare information technology software applications do not afford to annotate, comment or collaborate on specific patient information. Current systems allow for communication via email, whereby screen captures with annotations are attached to the email. Unfortunately, email systems are not integrated with EMR applications so that the comment threads can be stored for historical reference. Systems for improved annotation, comment and collaboration would be highly desirable. Systems allowing discussion threads and annotations to be stored with an EMR would also be highly desirable. The process is time-consuming, inefficient and impairs productivity of the medical professionals. In results, healthcare delivery if costly and valuable time is lost to provide medical services to a needy patient.

U.S. Patent Application Publication No. 2012/0198361 teaches a networked computer system which provides seamless navigation among a plurality of web applications. The networked computer system includes a server serving a plurality of applications and a client-side computer system connected to the server over a network. The client-side computer system includes a browser configured to access the plurality of applications. The browser includes a plurality of frames, each executing an interface configured to access a respective one of the plurality of applications over the network. The browser provides seamless navigation among the plurality of applications. The method includes steps of receiving a webpage including a plurality of interfaces to a plurality of applications, rendering the webpage within a browser, and seamlessly navigating from a first one of the interfaces to a second one of the interfaces in response to a user selection. Seamless navigation may be affected by hiding the first interface while un-hiding the second interface. A computer interface provides for seamless integration of a plurality of web applications to a web browser which provides access to a plurality of web applications in a plurality of frames, and which provides for seamless navigation from one web application accessed in one frame to another web application accessed in another frame. Large enterprise software systems often include numerous enterprise applications. In some cases, enterprise software systems include so many enterprise applications that it has become very difficult to determine where one application, e.g., enterprise resource planning (ERP), ends and another begins, e.g., supply chain management (SCM), product lifecycle management (PLM), customer relationship management (CRM) and enterprise asset management (EAM).

The inventors hereby incorporate the above-referenced patents and patent application publications into their specification.

### SUMMARY OF THE INVENTION

The present invention is a computing device implemented method includes the step of using a third-party disparate dental imaging system with capabilities to directly control a dental intraoral x-ray sensor imaging device via enacting communication with that specific brand of dental intraoral x-ray sensor imaging device for the purpose of acquiring new dental intraoral x-ray images of a patient's dental anatomy.

In a first aspect of the present invention is the computing device implemented method also includes the step of using a decoupled software application that is not part of the third-party dental imaging software.

In a second aspect of the present invention the decoupled software further contains an algorithm that detects when a specific brand or version of third-party dental imaging software is enacted upon the same computing device as the decoupled software application is executing.

In a third aspect of the present invention the computing device implemented method automates acquisition of images from non-supported dental imaging devices into closed architecture dental imaging software.

Other aspects and many of the attendant advantages will be more readily appreciated as the same becomes better understood by reference to the following detailed description and considered in connection with the accompanying drawings in which like reference symbols designate like parts throughout the figures.

### DESCRIPTION OF THE DRAWINGS

FIG. 1 is a conceptual diagram of a networking system including a desktop computer, a laptop computer, a server, a server, a network, a server, a tablet device and a private network group according to U.S. Patent Application Publication No. 2013/0226993.

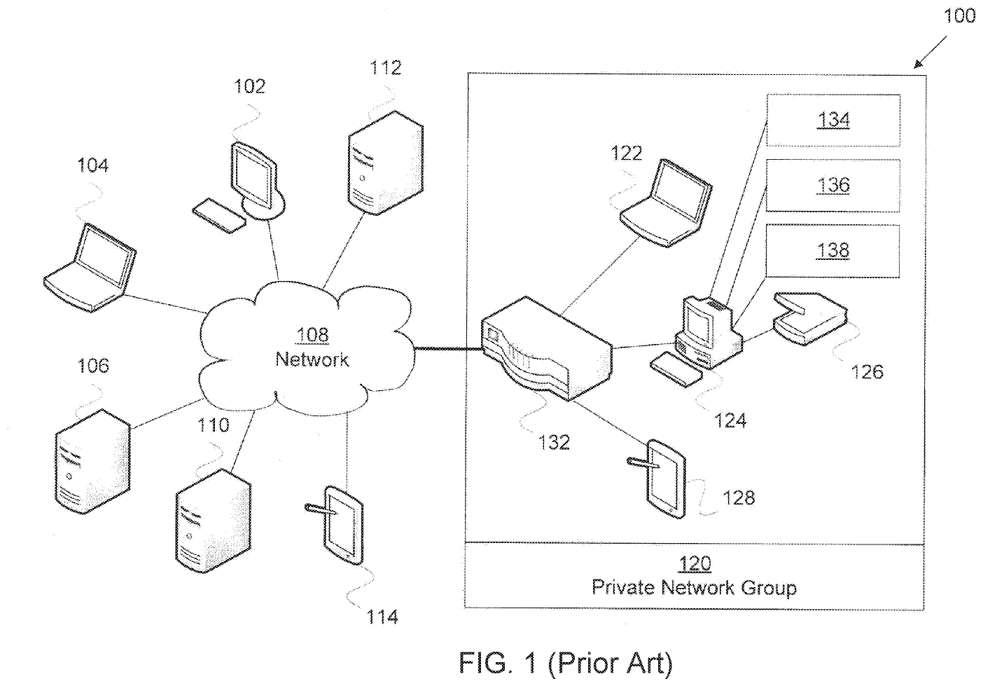

FIG. 2 is a conceptual diagram of a cloud-based server engine of the networking system of FIG. 1.

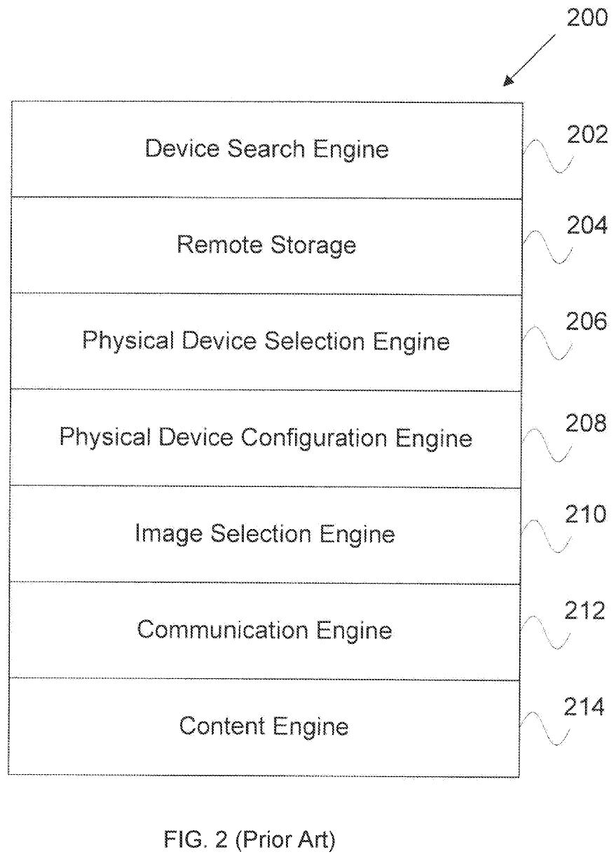

FIG. 3 is a conceptual diagram of a cloud-based client coupled to the networking system of FIG. 1.

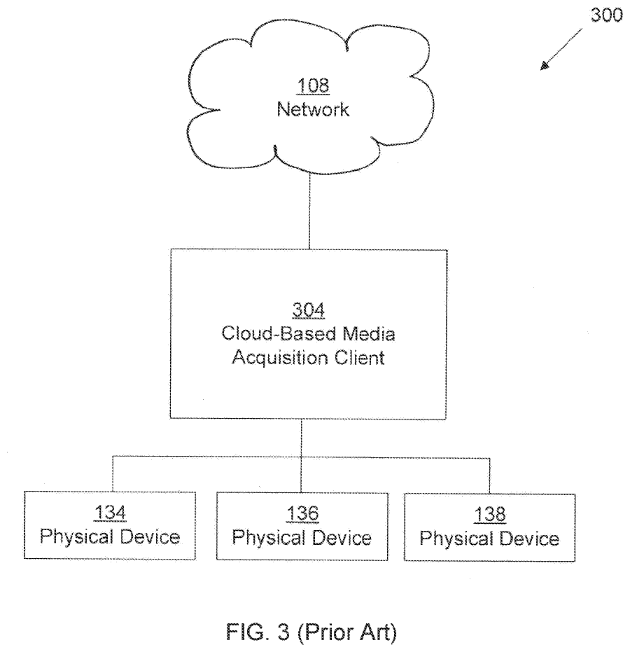

FIG. 4 is a schematic diagram of a universal image capture manager according to U.S. Patent Application Publication No. 2011/0304740.

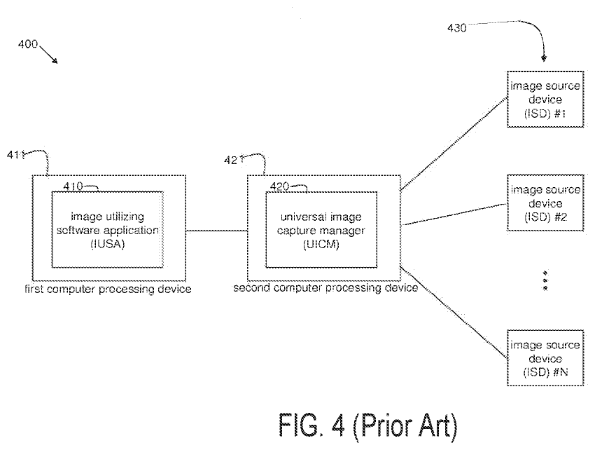

FIG. 5 is a schematic diagram of a software module architecture used in the universal image capture manager of FIG. 4.

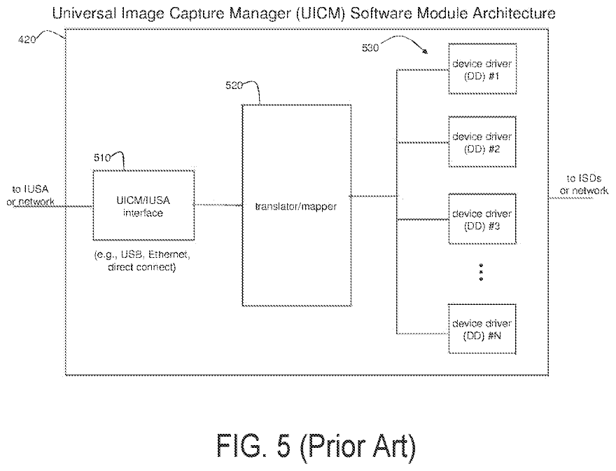

FIG. 6 is a schematic diagram of a dental office that uses proprietary or legacy dental imaging software and which is integrated to an originally supported imaging device but is not capable of integrating with an originally unsupported imaging device and is not using the claimed invention in U.S. Patent Application Publication No. 2017/0168812.

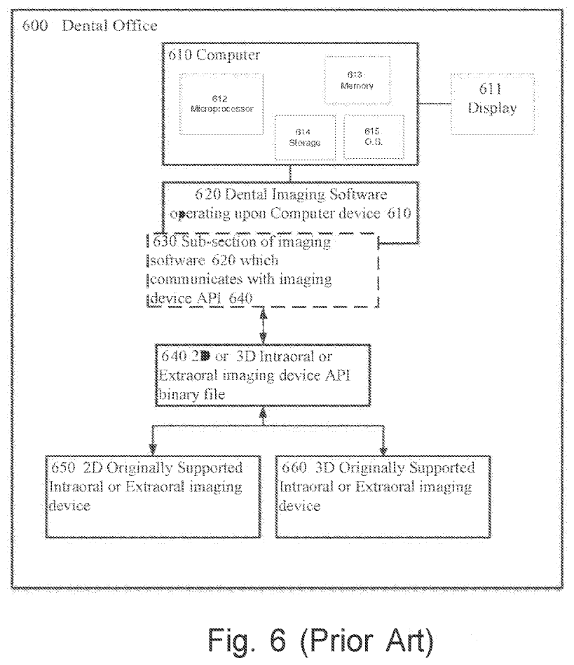

FIG. 7 is a schematic diagram of a dental office that uses proprietary or legacy dental imaging software and which is capable of integrating with an originally unsupported imaging device according to U.S. Patent Application Publication No. 2017/0168812.

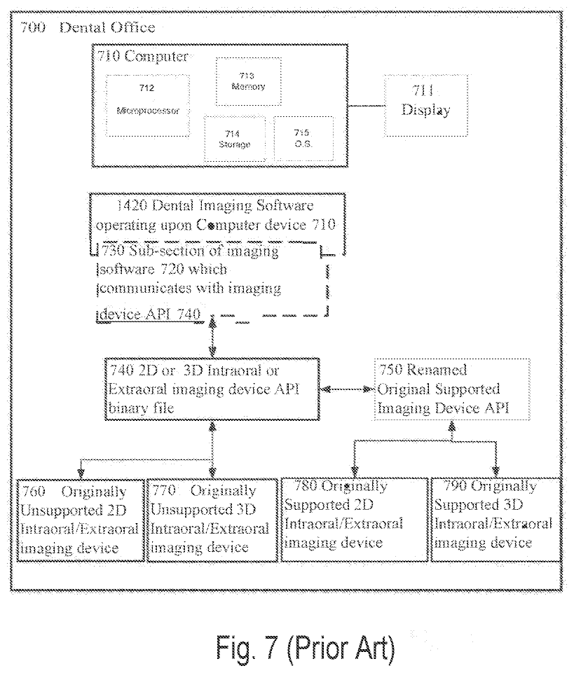

FIG. 8 is a schematic diagram of a flowchart of a method that integrates originally unsupported imaging devices into either legacy or proprietary dental imaging software according to U.S. Patent Application Publication No. 2017/0168812 an originally unsupported imaging device according to U.S. Patent Application Publication No. 2017/0168812.

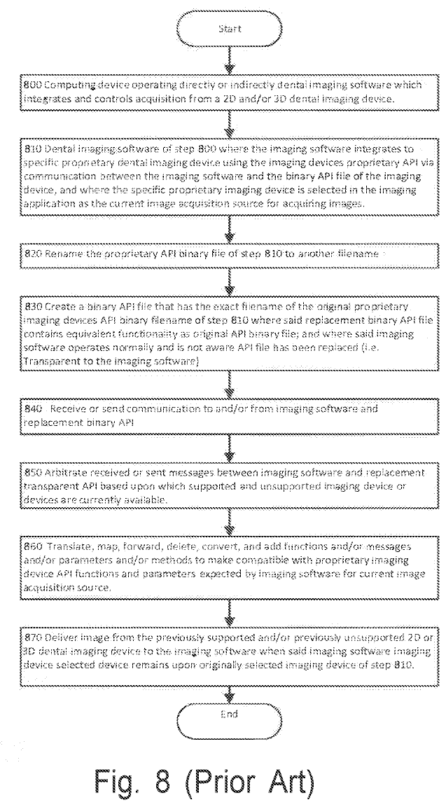

FIG. 9 is a schematic diagram of a single user interface which integrates disparate information technology applications and platforms across an enterprise in accordance with U.S. Pat. No. 6,041,362.

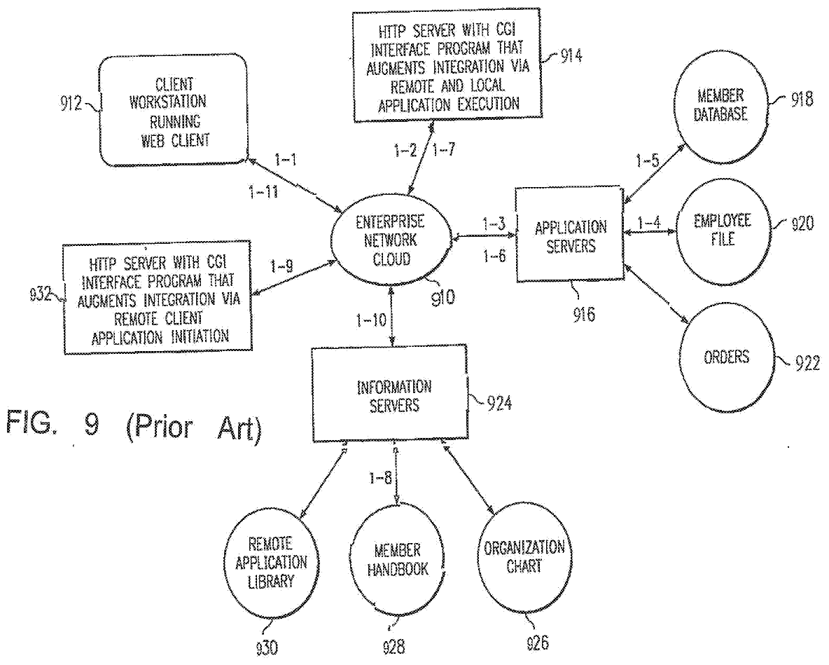

FIG. 10 is a block diagram of a dental imaging system including a closed-architecture dental imaging software, a decoupled monitoring, image acquisition, and automation software, and specific brands/models of dental intraoral x-ray imaging devices according to the present invention.

FIG. 11A is the first half of a flowchart that illustrates data and process flow according to the present invention.

FIG. 11B is the second half of a flowchart that illustrates data and process flow according to the present invention.

### DESCRIPTION OF THE PREFERRED EMBODIMENT

The scope of the invention relates to automating acquisition of dental intraoral digital x-ray images into a closed architecture dental imaging software system. The method includes the steps of a decoupled software application detecting when a specific brand or version of a third-party dental imaging software is operating upon the PC. The method includes the steps of a decoupled software monitoring the computing device checking for execution of a specific brand of third-party dental imaging software and when a user has enacted the acquisition process of acquiring a new intraoral x-ray image from a specific brand or model of dental intraoral x-ray sensor imaging device for which that detected dental imaging software is configured/selected to acquire images from. The method includes the steps of detecting what brands and models of digital intraoral x-ray sensors are attached and available for acquisition from the current PC. The method includes steps of programmatically communicating from a decoupled software to the dental imaging software commands or information that causes the user enacted acquisition process to be cancelled. The method includes the steps of the dental imaging software's acquisition graphical user interface being hidden/not displayed within the dental imaging software when the user interface of the decoupled software acquisition GUI is enacted. The method includes programmatic communication from the decoupled application software to instruct the dental imaging software to cancel and hide the graphical user interface of the enacted acquisition process within the third-party dental imaging software. The method includes the steps of the decoupled software displaying an alternate user interface that at a minimum is overlaid upon the displayed area on a display device where the imaging software was displayed when the user attempted to enact an acquisition from within that third-party dental image software. The method may include the step of detecting programmatically which brands and models of dental imaging device sensors are currently attached and available for acquisition and displaying the decoupled software user interface only when specific brands/models of sensors are detected. The method may include the steps of not displaying the user interface when a specific brand/model of sensor is connected/available on the PC. One of the uses of the alternate user interface is for the purpose of hiding and/or the alternate user interface being displayed at a minimum at least partially on top of the third-party dental imaging software. The method includes steps of acquiring images in the decoupled software application from a dental intraoral imaging sensor device. The method includes the steps of saving an image(s) to non-volatile storage and adding image metadata information to the image or an associated digital file. The method includes the steps of programmatically sending communication or data to the third-party dental imaging software and which step of communication enacts functionality in the third-party imaging software of adding an image to their internal patient record/database. The method includes the steps of providing the contents of the image saved on hard drive to the third-party dental imaging software via programmatic means. The method may include the steps of positioning the cursor of the operating system in an area over or intersecting with the dental imaging software graphical user interface. The method also includes associating metadata including at a minimum tooth information to the image or associated image data on non-volatile storage or in volatile memory and optionally date and time image was acquired in the decoupled software application. The method includes steps of programmatically making the image(s) acquired by the decoupled software available to the third-party dental imaging software via the decoupled software application causing an event or function to occur within the third-party dental imaging software via not using a public API of the dental imaging software; and which event/function results in adding the image(s) an image metadata which was programmatically communicated into the third-party dental imaging software and resulted in updating the dental imaging software image database and/or image data records. The method includes the steps of programmatically clearing/hiding the user interface of the decoupled software when acquisition of new images has completed within the decoupled software application and images have been transferred to the third-party dental imaging software. The method includes the steps of the third-party dental imaging software displaying the newly added image(s) and which image(s) have been programmatically associated with a patient's record in the third-party imaging software via actions of the decoupled software enacting the dental imaging software internal functionality. The method includes that images added to the dental imaging software via the decoupled software also enacts methods to associate each image with tooth numbers that were generated in the decoupled software and which were communicated into the dental imaging software via the programmatic decoupled software via possible methods of image attributes and/or remotely enacting functionality in the third-party dental imaging software via means of causing an event or function to be enacted within the third-party dental imaging software that associates teeth numbers with the image(s) via communicated metadata or information provided by the decoupled software. The method includes an algorithm for encoding tooth number metadata using a proprietary format that includes using relationships of dental tooth numbers in bitewing intraoral x-ray images.

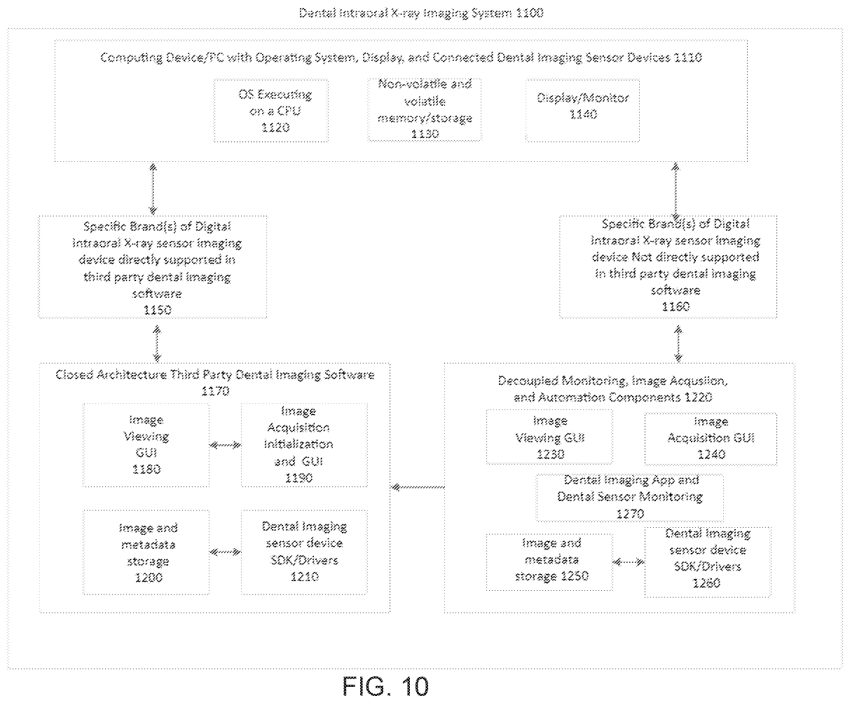

Referring to FIG. 10 a dental intraoral x-ray imaging system 1100 includes a computing device/PC with operating system, display and connected dental imaging sensor devices 1110. These devices 1110 include an OS executing on a CPU 1120, a non-volatile and volatile memory/storage 1130 and a display/monitor 1140. Either specific brands of digital intraoral x-ray sensor imaging device are directly supported in a third party dental imaging software 1150 or specific brands of digital intraoral x-ray sensor imaging device are not directly supported in a third party dental imaging software 1160. A closed architecture third party dental imaging software 1170 is coupled to the third party dental imaging software 1150. The closed architecture third party dental imaging software 1170 includes an image viewing GUI 1180, an image acquisition initialization and GUI 1190 which is bi-directionally coupled to the image viewing GUI 1180, an image and metadata storage 1200, and a dental imaging sensor device SDK/drivers 1210 which is bi-directionally coupled to the image and metadata storage 1200. Decoupled monitoring, image acquisition, and automation components are coupled to the third party dental imaging software 1160. The decoupled monitoring, image acquisition, and automation components 1220 includes an image viewing GUI 1230 and an image acquisition GUI 1240 which is bi-directionally coupled to the image viewing GUI 1230. The decoupled monitoring, image acquisition, and automation components 1220 also include an image and metadata storage 1250 and a dental imaging sensor device SDK/drivers 1260 which is bi-directional coupled to the image and metadata storage 1250. The decoupled monitoring, image acquisition, and automation components 1220 are further includes a dental imaging app and dental sensor monitoring 1270.

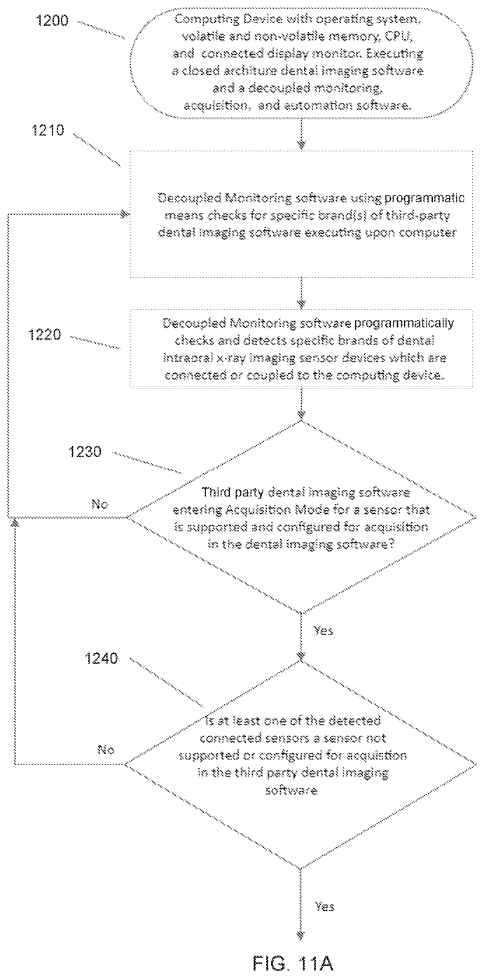

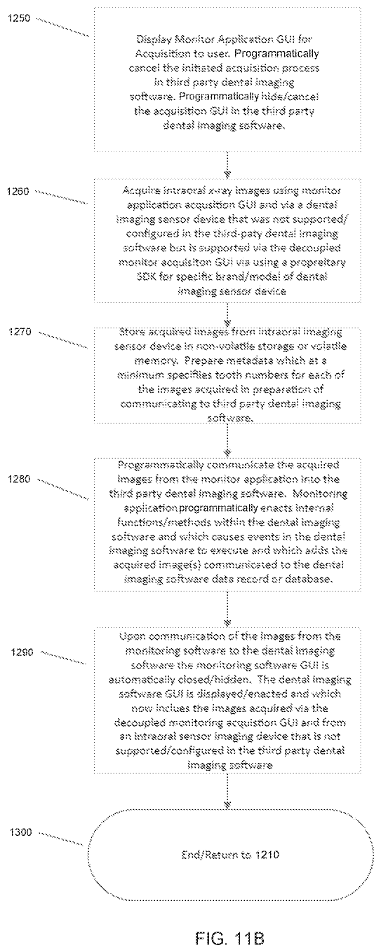

Referring to FIG. 11A in conjunction with FIG. 10 the operating method of the dental intraoral x-ray imaging system 1100 consists of the following steps. In Step 2100 the computing device/PC 1110 with an operating system, volatile and non-volatile memory, CPU, and connected display monitor executes a closed architecture dental imaging software and a decoupled monitoring, acquisition, and automation software. In Step 2110 a decoupled monitoring software uses programmatic means to check for a specific brand of a third-party dental imaging software executing upon computer. In Step 2120 the decoupled monitoring software programmatically checks and detects specific brands of dental intraoral x-ray imaging sensor devices which are connected or coupled to the computing device. In Step 2130 a third party dental imaging software enters acquisition mode for a sensor that is supported and configured for acquisition in the dental imaging software. In Step 2140 it is determined if at least one of the detected connected sensors a sensor not supported or configured for acquisition in the third party dental imaging software.

Still referring to FIG. 11A in conjunction with FIG. 10 in Step 2100 the dental intraoral x-ray imaging system of FIG. 10 is executing a specific brand of third-party closed architecture dental imaging software, and at a minimum simultaneously executing the decoupled monitoring app. One or more specific brands or models of dental intraoral x-ray imaging sensor devices are coupled to the computing device/PC. In Step 2110 the decoupled monitoring software using programmatic means detects a specific brand and/or version of third-party dental imaging software is operating upon the computing device. It is to be noted that Step 2110 can be combined with either S 2120 or Step 2130. In Step 2120 the decoupled monitoring software programmatically detects what dental intraoral x-ray sensor imaging devices are coupled to the computing device. In Step 2130 an algorithm in the decoupled software detects if the acquisition process and GUI is being enacted or has been enacted for a sensor that is supported and configured for acquisition within the third-party closed architecture dental imaging software. In Step 2140 the decoupled monitoring software returns to Step 2110 if no sensors were detected that are not supported or configured for acquisition by the third-party dental imaging software. In Step 2140 if an intraoral x-ray sensor imaging device is detected that is not configured or supported in the third-party dental imaging software.

Referring to FIG. 11B in conjunction with FIG. 10 and FIG. 11A in Step 2150 the operating program flow continues to from Step 2140 to Step 2150 the decoupled software displays an alternate intraoral x-ray acquisition GUI to the user and programmatically hides/cancels the acquisition enacted in the third-party dental imaging software, including deactivation of the SDK for the sensor configured for use in the third-party dental imaging software. In Step 2160 the software displays and enacts an acquisition GUI allowing acquisition from the non-supported sensors that are coupled to the computing device/PC and detected in Step 2120. In Step 2170 the images acquired and stored images have metadata added to each image and at a minimum includes tooth numbers. In Step 2180 the decoupled software or associated acquisition components remotely and programmatically communicate with the third-party closed architecture dental imaging software to enact internal functions/methods within the third-party dental imaging software that cause events which adds the communicated images and metadata which at a minimum includes tooth numbers to the dental imaging software database or data records. In Step 2190 the decoupled software enacts hiding/closing of the acquisition GUI upon completion of programmatic communication of the images acquired from the sensor not supported in the third-party dental imaging software. The images communicated can include tooth numbers added to the headers of the image format. Specific area of the image format header may be utilized for storing tooth information. The tooth numbers may use a specific format including concatenating a range of tooth numbers into a single integer number and storing in the header. Bitewing tooth range for USA tooth numbers for one of the bitewings is 1, 2, 3, 30, 31, and 32 and would be integer 132. Tooth numbers 7, 8, and 9 would be 79. Tooth numbers 10, 11, 12, 13, and 14 would be 1014. Tooth numbers may use USA tooth numbers, FDI international tooth numbers, Palmer notation, or custom alpha and/or numeric format. Each image communicated may include tooth numbers representing the teeth present in that specific image. The image format may include an integer number of concatenated tooth numbers. The algorithm for concatenated tooth numbers uses known dental anatomy tooth relationships for images that include non-contiguous tooth number such as bitewing. The data representing the tooth numbers may be tooth numbers in USA format, FDI format, Palmer notation, or a custom alpha and/or numerical format. Images acquired in the alternate GUI are automatically associated with one or more tooth numbers prior to the images being communicated to the disparate imaging system. Images acquired in the alternate GUI are associated with one or more tooth numbers via user interaction with the alternate GUI and prior to the images being communicated to the disparate imaging system.

Referring again to FIG. 10 in conjunction with FIG. 11A and FIG. 11B a computing device implemented method 2100 includes the steps of using a third-party disparate dental imaging system with capabilities to directly control a dental intraoral x-ray sensor imaging device via enacting communication with that specific brand of dental intraoral x-ray sensor imaging device for the purpose of acquiring new dental intraoral x-ray images of a patient's dental anatomy and using a decoupled software application that is not part of the third-party dental imaging software and which decoupled software contains an algorithm that detects when a specific brand or version of third-party dental imaging software is enacted upon the same computing device as the decoupled software application is executing. The computing device implemented method 2100 also includes the steps of using a the decoupled software application algorithm to programmatically detect when a user of that specific dental imaging software enacts a new acquisition from an intraoral x-ray imaging device via the dental imaging software using that devices proprietary SDK, using the decoupled software algorithm to programmatically cancel the user enacted acquisition of the dental imaging software and including communications that result in deactivating the enacted SDK that is being executed via the third-party disparate dental imaging software, using the decoupled software algorithm to programmatically hide the acquisition GUI of the third-party dental imaging software, using the decoupled software algorithm upon detection of the user enacted acquisition in the third-party dental imaging software to automatically display an alternate acquisition GUI to the user for the purpose of acquiring images from a dental intraoral x-ray imaging device that is not supported/configured within the third-party dental imaging software application executing on the computing device, using the decoupled software application to acquire images from the specific brand of dental intraoral x-ray imaging device via using that sensors proprietary SDK; and which SDK is not loaded/enacted within the third-party dental imaging software, using the decoupled software application to programmatically make images) acquired by the decoupled software available to the third-party dental imaging software via the decoupled software application programmatically causing an event or function to occur within the third-party dental imaging software; and which event/function results in adding the images) an image metadata which was programmatically communicated into the third-party dental imaging software and resulted in updating the dental imaging software image database and/or image data records, and using the decoupled software application, upon acquiring and transferring images to the third-party dental imaging software, to automatically hide the alternate GUI that was enacted to acquire images from the dental intraoral x-ray imaging sensor device that is unsupported in the third-party dental imaging software. The algorithm does not use a public API to communicate with the third-party dental imaging software. The algorithm positions the operating system cursor at coordinates that intersect with display coordinates for the third-party dental imaging software GUI.

Referring again to FIG. 10 in conjunction with FIG. 11A and FIG. 11B a computing device implemented method 2100 includes the steps of using a third-party disparate dental imaging system with capabilities to directly control a dental intraoral x-ray sensor imaging device via enacting communication with that specific brand of dental intraoral x-ray sensor imaging device for the purpose of acquiring new dental intraoral x-ray images of a patient's dental anatomy, using a decoupled software application that is not part of the third-party dental imaging software and which decoupled software contains an algorithm that detects when a specific brand or version of third-party dental imaging software is enacted upon the same computing device as the decoupled software application is executing, using the decoupled software algorithm upon user interaction to display an alternate acquisition GUI to the user for the purpose of acquiring images from a dental intraoral x-ray imaging device that is not supported/configured within the third-party dental imaging software application executing on the computing device, using the decoupled software application to acquire images from the specific brand of dental intraoral x-ray imaging device via using that sensors proprietary SDK; and which SDK is not loaded/enacted within the third-party dental imaging software, using the decoupled software application to programmatically make images) acquired by the decoupled software available to the third-party dental imaging software via the decoupled software application programmatically causing an event or function to occur within the third-party dental imaging software; and which event/function results in adding the images) an image metadata which was programmatically communicated into the third-party dental imaging software and resulted in updating the dental imaging software image database and/or image data records, and using the decoupled software application, upon acquiring and transferring images to the third-party dental imaging software, to automatically hide the alternate GUI that was enacted to acquire images from the dental intraoral x-ray imaging sensor device that is unsupported in the third-party dental imaging software.

Referring still again to FIG. 10 in conjunction with FIG. 11A and FIG. 11B a computing device implemented method 2100 includes the steps of executing a third-party disparate dental imaging system with capabilities to directly control a dental intraoral x-ray sensor imaging device via enacting communication with that specific brand of dental intraoral x-ray sensor imaging device for the purpose of acquiring new dental intraoral x-ray images of a patient's dental anatomy, simultaneously executing a decoupled software application that is not part of the third-party dental imaging software and which decoupled software contains an alternate GUI for acquiring images from a dental intraoral x-ray sensor imaging device that it not supported or configured in the disparate dental imaging system, acquiring images from the intraoral x-ray sensor device coupled to the alternate GUI acquisition and which process includes enacting the intraoral x-ray imaging device proprietary SDK, via programmatic means or user interaction assigning one or more teeth to the images) acquired in the alternate GUI where the tooth numbers) assigned typically represent the specific dental teeth anatomy present within each specific intraoral x-ray image, saving the acquired images and tooth information to the computing device in a format that supports image data and non-image data header information, using an algorithm to create a single number that represents the collection of teeth numbers assigned to each specific image where the tooth number creation algorithm concatenates the first or lowest tooth number selected in a group of contiguous teeth and the last tooth number or highest tooth number selected in the group of teeth and where the tooth number creation algorithm also uses dental intraoral anatomy tooth number relationships for creating a single number representing the specific teeth numbers for cases where the teeth numbers for the image are not all contiguous teeth such as with intraoral bitewing images.

From the foregoing a computing device implemented method for automatically making a diagnostic presentation of a machine learning/AI detected dental condition or dental feature to assist with diagnosis and/or feature identification has been described. It should be noted that the sketches are not drawn to scale and that distances between the figures are not to be considered significant.

Accordingly, it is intended that the foregoing disclosure and showing made in the drawing shall be considered only as an illustration of the principle of the present invention.

What is claimed is:

1. A computing device implemented method comprising the steps of:
a. using a third-party disparate dental imaging system with capabilities to directly control a dental intraoral x-ray sensor imaging device via enacting communication with that specific brand of dental intraoral x-ray sensor imaging device for the purpose of acquiring new dental intraoral x-ray images of a patient's dental anatomy; and
b. using a decoupled software application that is not part of the third-party dental imaging software and which decoupled software contains an algorithm that detects when a specific brand or version of third-party dental imaging software is enacted upon the same computing device as the decoupled software application is executing.

2. A computing device implemented method according to claim 1 also comprising the step of using the decoupled software application algorithm to programmatically detect when a user of that specific dental imaging software enacts a new acquisition from an intraoral x-ray imaging device via the dental imaging software using that devices proprietary SDK.

3. A computing device implemented method according to claim 1 also comprising the step of using the decoupled software algorithm to programmatically cancel the user enacted acquisition of the dental imaging software and including communications that result in deactivating the enacted SDK that is being executed via the third-party disparate dental imaging software.

4. A computing device implemented method according to claim 2 further comprising the step of using the decoupled software algorithm to programmatically hide the acquisition GUI of the third-party dental imaging software.

5. A computing device implemented method according to claim 2 also comprising the step of:
a. using the decoupled software algorithm upon detection of the user enacted acquisition in the third-party dental imaging software to automatically display an alternate acquisition GUI to the user for the purpose of acquiring images from a dental intraoral x-ray imaging device that is not supported/configured within the third-party dental imaging software application executing on the computing device;
b. using the decoupled software application to acquire images from the specific brand of dental intraoral x-ray imaging device via using that sensors proprietary SDK; and which SDK is not loaded/enacted within the third-party dental imaging software;
c. using the decoupled software application to programmatically make images) acquired by the decoupled software available to the third-party dental imaging software via the decoupled software application programmatically causing an event or function to occur within the third-party dental imaging software; and which event/function results in adding the images) an image metadata which was programmatically communicated into the third-party dental imaging software and resulted in updating the dental imaging software image database and/or image data records; and
d. using the decoupled software application, upon acquiring and transferring images to the third-party dental imaging software, to automatically hide the alternate GUI that was enacted to acquire images from the dental intraoral x-ray imaging sensor device that is unsupported in the third-party dental imaging software.

6. A computing device implemented method according to claim 4 wherein the algorithm does not use a public API of the disparate 3rd party imaging software to communicate with the third-party dental imaging software.

7. A computing device implemented method according to claim 4 wherein the algorithm positions the operating system cursor at coordinates that intersect with display coordinates for the third-party dental imaging software GUI.

8. A computing device implemented method which includes the steps of:
a. using a third-party disparate dental imaging system with capabilities to directly control a dental intraoral x-ray sensor imaging device via enacting communication with that specific brand of dental intraoral x-ray sensor imaging device for the purpose of acquiring new dental intraoral x-ray images of a patient's dental anatomy;
b. using a decoupled software application that is not part of the third-party dental imaging software and which decoupled software contains an algorithm that detects when a specific brand or version of third-party dental imaging software is enacted upon the same computing device as the decoupled software application is executing; and
c. using the decoupled software algorithm upon user interaction to display an alternate acquisition GUI to the user for the purpose of acquiring images from a dental intraoral x-ray imaging device that is not supported/configured within the third-party dental imaging software application executing on the computing device;
d. using the decoupled software application to acquire images from the specific brand of dental intraoral x-ray imaging device via using that sensors proprietary SDK; and which SDK is not loaded/enacted within the third-party dental imaging software;
e. using the decoupled software application to programmatically make images acquired by the decoupled software available to the third-party dental imaging software via the decoupled software application programmatically causing an event or function to occur within the third-party dental imaging software; and which event/function results in adding the images an image metadata which was programmatically communicated into the third-party dental imaging software and resulted in updating the dental imaging software image database and/or image data records; and
f. using the decoupled software application, upon acquiring and transferring images to the third-party dental imaging software, to automatically hide the alternate GUI that was enacted to acquire images from the dental intraoral x-ray imaging sensor device that is unsupported in the third-party dental imaging software.

9. A computing device implemented method comprising the steps of:
a. executing a third-party disparate dental imaging system with capabilities to directly control a dental intraoral x-ray sensor imaging device via enacting communication with that specific brand of dental intraoral x-ray sensor imaging device for the purpose of acquiring new dental intraoral x-ray images of a patient's dental anatomy;
b. simultaneously executing a decoupled software application that is not part of the third-party dental imaging software and which decoupled software contains an alternate GUI for acquiring images from a dental intraoral x-ray sensor imaging device that it not supported or configured in the disparate dental imaging system;
c. acquiring images from the intraoral x-ray sensor device coupled to the alternate GUI acquisition and which process includes enacting the intraoral x-ray imaging device proprietary SDK via programmatic means or user interaction;
d. assigning one or more teeth to the images acquired in the alternate GUI and where the tooth numbers assigned typically represent the specific dental teeth anatomy present within each specific intraoral x-ray image;
e. saving the acquired images and tooth information to the computing device in a format that supports image data and non-image data header information; and
f. using an algorithm to create a single number that represents the collection of teeth numbers assigned to each specific image wherein the tooth number creation algorithm concatenates the first or lowest tooth number selected in a group of contiguous teeth and the last tooth number or highest tooth number selected in the group of teeth and wherein the tooth number creation algorithm also uses dental intraoral anatomy tooth number relationships for creating a single number representing the specific teeth numbers for cases where the teeth numbers for the image are not all contiguous teeth such as with intraoral bitewing images.
